(19) United States
(12) Patent Application Publication
(10) Pub. No.: US 2004/0067218 A1
(43) Pub. Date: Apr. 8, 2004

(54) Extramedullary adipose tissue cells and use thereof for regenerating hematopoietic and muscular tissue

(76) Inventors: Louis Casteilla, Escalouens (FR); Beatrice Cousin, Toulouse (FR); Luc Penicaud, Toulouse (FR); Mireille Andre, Blagnac (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/250,548

(22) PCT Filed: Jan. 10, 2002
(86) PCT No.: PCT/FR02/00071

(30) Foreign Application Priority Data
Jan. 10, 2001 (FR) 01/00249

(51) Int. Cl. A61K 45/00; C12N 5/08

(52) U.S. Cl. 424/93.7; 435/366

(57) ABSTRACT

The invention concerns cells derived from the cellular fraction of the vascular stroma of the extramedullary adipose tissue, methods for preparing them and their use in regeneration of hematopoietic lines and cardiac and skeletal muscular tissues, in particular for treating genetic or acquired hemopathies, myopathies and cardiomypathies.

The present invention relates to cells derived from extramedullary adipose tissue, to methods for the preparation thereof, and also to uses thereof for regenerating hematopoietic lines, in particular for the treatment of genetic or acquired hemopathies (cancer, chemotherapy, irradiation), and for regenerating cardiac or skeletal muscle, in particular for the treatment of myopathies, cardiomyopathies and diseases associated with muscle degeneration (myocardial infarction).

Existing means for regenerating hematopoietic lines essentially comprise:

bone marrow transplantation, and

transplantation of stem cells or of hematopoietic precursors, isolated from hematopoietic tissues.

These means have the following drawbacks:

bone marrow transplantation depends on the existence of a compatible donor and is relatively inefficient due to the high percentage of transplant rejection, related to contamination of the transplant with the recipient's lymphocytes (graft versus host, or GvH, reaction);

transplantation of purified stem cells or hematopoietic precursors (CD34+, Thy1+, Lin⁻), which also depends on the existence of a compatible donor, makes it possible to avoid the failures related to transplant rejections. However, due to the limited capacities for expansion of these cells, it is difficult to obtain a sufficient amount of cells for an efficient transplant.

In order to regenerate skeletal muscle tissues, the use of skeletal muscle precursors (satellite cells) or of bone marrow has for a long time been envisioned. However, due to the limited capacities for expansion of these cells, it is difficult to obtain a sufficient amount of cells for an efficient transplant.

The regeneration of cardiac muscle tissue (myocardium) has been envisioned quite recently, given that, unlike skeletal muscles, the heart does not possess reservoirs of precursor cells. To regenerate the myocardium, it has been proposed to transplant autologous satellite cells into the heart. However, this approach is not satisfactory insofar as these transplanted satellite cells differentiate into skeletal muscle cells which do not have the same contractile characteristics as cardiomyocytes.

Consequently, a real need exists for novel means, and in particular for cells, able to regenerate the various hematopoietic lines and to regenerate the myocardium and skeletal muscle, which are more efficient and simpler to use than the means of the prior art.

It has recently been shown that stem cells of certain nonhematopoietic tissues are capable of regenerating all of the hematopoietic lines of mice, which have been lethally irradiated, namely:

muscle stem cells derived from adult mouse skeletal muscle expressing:

either markers common to all stem cells (Sca-1, c-Kit) but not the CD45 marker, which is specific for hematopoietic stem cells (Jackson et al., PNAS, 1999, 96, 14482-14486),

or the receptor for bone marrow morphogenetic protein type 2 (BMP2), (Pang, Blood, 2000, 95, 1106-1108), and

neural stem cells derived from adult mouse brain (Bjornson et al., Science, 1999, 283, 534-537).

Similarly, to obtain cardiomyocytes, it has been proposed to use human embryonic cells, bone marrow mesenchymal cells or endothelial cells (Kehat et al., J. Clin. Invest., 2001, 108, 407-414; Muller et al., FASEB J., 2000, 14, 2540-2548, Toma et al., Circulation, 2002, 105, 93-98; Liechty et al., Nat. Medicine, 2000, 11, 1282-1286; Condorelli et al., PNAS, 2001, 98, 10733-10738; Wang et al., J. Thorac. Cardiovasc. Surg 2001, 122, 699-705; Jackson et al., J. Clin. Invest., 2001, 107, 1395-1402).

Two hypotheses have been put forward to explain these results: stem cells close to the totipotency of embryonic cells will persist in adult tissues (brain, muscle, etc.) and will be capable of differentiating into various cell types, or else specialized stem cells of these tissues will possess very great plasticity and will be capable of dedifferentiating or being reprogrammed (transdifferentiation).

These results have important consequences for the treatment of functional deficiencies of the bone marrow (medullary aplasia), the treatment of contamination of the bone marrow with tumor cells (neuroblastoma), and for the correction of genetic abnormalities of hematopoietic cells (genetic manipulation of the hematopoietic tissue). These results also have consequences for the treatment of functional muscle deficiencies (myopathies and cardiomyopathies) and of diseases associated with muscle degeneration (myocardial infarction).

In fact, the use of stem cells or of precursors of nonhematopoietic tissues will make it possible to avoid the problems associated with bone marrow transplant rejection or with an insufficient amount of CD34 cells, insofar as the regeneration of hematopoietic lines of a sick adult individual, by transplanting autologous nerve or muscle tissue taken from this same individual, could be envisioned. Similarly, the use of stem cells other than satellite cells would make it possible to regenerate the myocardium by transplanting bone marrow cells, embryonic cells or endothelial cells.

However, in practice, efficient regeneration of hematopoietic lines and regeneration of cardiac and skeletal muscle tissues from the abovementioned cells is difficult to perform, owing to the technical difficulties of sampling and the small amounts of available tissues. Added to these technical difficulties are also ethical problems associated with the use of embryonic tissues.

Consequently, the inventors have given themselves the aim of providing cells able to regenerate hematopoietic lines and muscle tissues in a long-lasting manner, which are isolated from tissues easy to sample and available in large amounts.

Adipose tissue exists in various forms in mammals: white adipose tissue which represents the main storage organ of the body, thermogenic brown adipose tissue, and medullary adipose tissue, the exact role of which is not known.

This adipose tissue consists of two cellular fractions:

an adipocyte fraction comprising differentiated adipose cells: immature adipocytes (small adipose cells) and mature adipocytes which represent 30% to 60% of the cells of adipose tissue. This cellular fraction is characterized by the accumulation of triglycerides and the expression of late and very late markers such as GPDH (glycerol-3-phosphate dehydrogenase), and

a non-adipocyte fraction, called vascular fraction of stroma, comprising some blood cells, endothelial cells, pericytes, fibroblasts and adipocyte precursors, in particular preadipocytes characterized by the absence of lipids in their cytoplasm and the expression of early markers such as the α2 chain of collagen VI (A2COL6/pOb24) and lipoprotein lipase (LPL).

These two cellular fractions can be separated by their difference in density, according to methods such as those described by Bjorntorp et al. (J. Lipid Res., 1978, 19, 316-324).

Conventionally, adipocyte differentiation is illustrated schematically in the following way:

multipotent stem cell→adipocyte precursor or preadipocyte→immature adipocyte→mature adipocyte.

The very early precursors, namely multipotent stem cells capable of engendering various types of mesenchymal cells such as adipocyte cells and muscle cells, have not been identified. However, it has been shown that mesenchymal cell lines are capable of differentiating into adipocytes, chondrocytes or fibroblasts, spontaneously (teratocarcinoma line T984) or after treatment with 5-azacytidine (10T1/2, 3T3, CHEF-18).

Adipocyte precursors have been cloned from these lines (clonal line 1246), from mouse embryo (3T3-L1, 3T3-F442A, A31T, TA1) or from hamster embryo (CHEF-18) or else from adult mouse (Ob17, HFGu, BFC-1, ST13, MS3-2A, MC 3t3-G2/PA6). These adipocyte precursors are capable of differentiating into adipocytes, in vitro or in vivo (Ailhaud et al., Annu. Rev. Nutr., 1992, 12, 207-233).

In addition, in prior studies (Cousin et al., The FASEB Journal, 1999, 13, 305-312), the inventors, who were interested in the relationship between adipocytes and macrophages in obesity and the inflammatory response, showed that preadipocytes (primary cultures derived from the stroma-vascular fraction, or 3T3-L1 line), like macrophages, have a phagocytic activity and that the MOMA-2 marker, which is specific for monocyte-macrophages, is also expressed by preadipocytes and adipocytes. However, it emerges from that article that preadipocytes are different from macrophages insofar as, using conventional techniques, it is impossible to detect the presence, at the surface of these cells, of the F4/80 and Mac1 markers which are specific for mature macrophages.

Adipose tissue has a regeneration potential which persists throughout an individual's life and is associated with maintaining a population of preadipocytes within the various adipose deposits. In fact, the number of adipocytes present in a given deposit can vary in considerable proportions depending on the physiological or physiopathological conditions.

Thus, it has been shown that, in humans or in adult, even old, rodents, the cells of the stroma-vascular fraction of adipose tissue which comprise a large proportion of preadipocytes expressing early markers of differentiation (A2COL6/pOb24, LPL, IFG-1, etc.) are capable of proliferating in vitro and of differentiating into adipocytes (Ailhaud et al., 1992, mentioned above).

Thus, it has been proposed to use multipotent stem cells derived from the stroma-vascular fraction of adipose tissue to regenerate hematopoietic lines, and also nerve and hepatic tissues (international application WO 01/62901) or muscle, bone and cartilage tissues (Zuk et al., Tissue Eng., 2001, 7, 20, 211-228). However, the means described in those documents have not made it possible to isolate such stem cells effectively able to differentiate into functional cells capable of regenerating a hematopoietic, muscle, nerve or hepatic activity.

Surprisingly, the inventors have now shown that cells of the stroma-vascular fraction of extramedullary adipose tissue (or cellular fraction of the vascular stroma) are effectively capable of differentiating into hematopoietic lines and into cardiomyocytes; such cells are able to regenerate hematopoiesis in mammals, in particular in mice, which have been lethally irradiated, and to regenerate a functional heart, in particular when they are transplanted into the area of infarction, after a cardiac event.

Consequently, a subject of the present invention is the cellular fraction of the vascular stroma of extra-medullary adipose tissue, as a medicinal product; in fact, this fraction makes it possible, surprisingly, to regenerate hematopoietic lines and to regenerate cardiac muscle tissue (myocardium).

A subject of the invention is also the use of said fraction, for preparing a medicinal product intended for the treatment of diseases in which an induced or constitutive medullary depletion is observed, such as malignant hemopathies, bone marrow tumors, hemopathies of genetic or acquired origin, or disorders subsequent to irradiation or to chemotherapy.

A subject of the invention is also the use of said fraction, for preparing a medicinal product intended for the treatment of myopathies and cardiomyopathies of genetic or acquired origin, and pathological conditions (induced or constitutive) associated with muscle degeneration, such as myocardial infarction.

In fact, cells of the stroma-vascular fraction of extramedullary adipose tissue are capable of regenerating myeloid lines which engender monocytes/macrophages, polynuclear basophils, eosinophils and neutrophils, platelets and erythrocytes and/or lymphoid lines which engender T lymphocytes and B lymphocytes. These cells are also capable of differentiating into functional cardiomyocytes exhibiting contractile activity.

In accordance with the invention, the cellular fraction of the vascular stroma is isolated by difference in density, in particular according to the protocol described by Bjorntorp et al. (mentioned above).

A subject of the present invention is also isolated and purified cells able to regenerate hematopoietic lines, characterized in that they are isolated from the cellular fraction of the vascular stroma of extramedullary adipose tissue.

A subject of the present invention is also isolated and purified cells able to differentiate into cardiomyocytes, characterized in that they are isolated from the cellular fraction of the vascular stroma of extra-medullary adipose tissue.

According to an advantageous embodiment of said cells, they can be obtained by the following successive steps:

taking a sample of extramedullary adipose tissue,

isolating the cellular fraction of the vascular stroma, preferably by digestion of the extracellular matrix with proteolytic enzymes and by physical separation, in particular by difference in density, and

purifying the cells by physical separation and/or by immunoselection.

Advantageously:

the physical separation is carried out by difference in adhesion onto a suitable solid support or by difference in density (centrifugation in a suitable gradient, elutriation),

the immunoselection is carried out using at least one antibody specific for a marker expressed by the adipocyte, hematopoietic or cardiomyocyte precursors (positive selection) and/or at least one antibody specific for a marker absent from said precursors (negative selection), which are known per se to those skilled in the art.

According to an advantageous arrangement of this embodiment, the purification step is preceded by an additional step of culturing the cells in a semi-solid medium containing suitable growth factors and/or cytokines.

By way of nonlimiting example, mention may be made of a medium containing methylcellulose supplemented with fetal calf serum, bovine serum, insulin, transferrin, SCF (Stem Cell Factor), IL3 and IL6.

According to another advantageous embodiment of said cells able to regenerate hematopoietic lines, they express at least one marker for adipocyte stem cells or precursors and/or at least one marker for hematopoietic stem cells or precursors.

According to another advantageous embodiment of said cells able to differentiate into cardiomyocytes, they express at least one marker for adipocyte stem cells or precursors and/or at least one marker for cardiomyocyte stem cells or precursors.

In the context of the invention, the stem cells and the precursors correspond to multipotent cells which have properties of clonal expansion and of tissue differentiation, and these two terms are considered to be equivalent.

According to an advantageous arrangement of this embodiment of said cells able to regenerate hematopoietic lines and of said cells able to differentiate into cardiomyocytes, the marker for adipocyte precursors is selected from the group consisting of A2COL6/pOb24, LPL and Pref-1.

According to another advantageous arrangement of this embodiment of said cells able to regenerate hematopoietic lines, the marker for hematopoietic stem cells or precursors is selected from the group consisting of: CD34, CD45, Thy-1, Sca-1, CD117 and CD38.

According to yet another advantageous arrangement of this embodiment of said cells able to differentiate into cardiomyocytes, the marker for cardiomyocyte precursors is selected from the group consisting of α-actinin and the GATA-4 factor.

Cells able to regenerate hematopoietic lines in accordance with the invention consist in particular of cells which express at least A2COL6/pOb24, CD34 and CD45.

Preferably, said cells as defined above are of human origin.

A subject of the present invention is also modified cells, characterized in that they consist of cells as defined above which have been genetically modified.

According to an advantageous embodiment of said modified cells, they comprise at least one mutation of an autologous gene.

For the purpose of the present invention, the expression “mutation of a gene” is intended to mean an insertion, a deletion or a substitution of at least one nucleotide of said gene.

For example, genes of the MHC of said cells can be mutated in order to allow a heterologous transplant.

According to another embodiment of said modified cells, they contain at least one copy of a heterologous gene, in particular a gene of therapeutic interest. Advantageously, the product of said gene is secreted by said modified cells.

For example, said cells express an interleukin or a factor which acts on blood clotting.

In accordance with the invention, said modified cells are obtained according to techniques which are known per se to those skilled in the art; mention may in particular be made of homologous recombination, infection with a recombinant vector such as a recombinant virus (retrovirus, lentivirus, adenovirus or adenovirus-associated virus (AAV)) or transfection with a recombinant plasmid, which are described in Current Protocols in Molecular Biology, (1990-2000), John Wiley and Sons, Inc. New York. Depending on the nature of the recombinant vector, said heterologous gene of interest is integrated into the genome of said cells or else is present in extrachromosomal form.

Preferably, said modified cells as defined above are of human origin.

A subject of the present invention is also immortalized cell lines derived from the human cells as defined above.

In accordance with the invention, the immortalized cell lines are obtained by successive passages, as described in Green et al., Cell, 1974, 3, 127-133.

A subject of the present invention is also a medicinal product intended to regenerate hematopoietic lines, characterized in that it comprises cells (isolated and/or modified) able to regenerate hematopoietic lines or lines derived from these cells as defined above, and at least one pharmaceutically acceptable vehicle.

Advantageously, said medicinal product is administered parenterally, preferably intravenously.

A subject of the present invention is also a medicinal product intended to regenerate the myocardium, characterized in that it comprises cells (isolated and/or modified) able to differentiate into cardiomyocytes or lines derived from these cells as defined above, and at least one pharmaceutically acceptable vehicle.

Advantageously, said medicinal product is administered locally at the site of the lesion.

A subject of the present invention is also the use of the cells able to differentiate into hematopoietic lines or else of the modified cells or of the lines derived from these cells, as defined above, for preparing a medicinal product intended for the treatment of diseases in which induced or constitutive medullary depletion is observed.

A subject of the present invention is also the use of the cells able to differentiate into cardiomyocytes or else of the modified cells or of the lines derived from these cells, as defined above, for preparing a medicinal product intended for the treatment of cardio-myopathies and of diseases in which cardiac muscle degeneration is observed.

A subject of the present invention is also the use of the cells able to differentiate into cardiomyocytes or else of the modified cells or of the lines derived from these cells, as defined above, for screening molecules capable of modulating (activating or inhibiting) cardiac activity.

A subject of the present invention is also the use of the cells able to differentiate into hematopoietic lines or else of the modified cells or of the lines derived from these cells, as defined above, for screening molecules capable of modulating (activating or inhibiting) hematopoietic activity.

A subject of the present invention is also a method for preparing the isolated and purified cells able to regenerate hematopoietic lines, as defined above, which method is characterized in that it comprises at least the following steps:

a₁) taking a sample of extramedullary adipose tissue,

b₁) isolating the cellular fraction of the vascular stroma, preferably by digestion of the extra-cellular matrix with proteolytic enzymes and by physical separation, and

c₁) purifying the cells by physical separation and/or by immunoselection.

A subject of the present invention is also a method for preparing the isolated and purified cells able to differentiate into cardiomyocytes, as defined above, which method is characterized in that it comprises at least the following steps:

a₂) taking a sample of extramedullary adipose tissue,

b₂) isolating the cellular fraction of the vascular stroma, preferably by digestion of the extra-cellular matrix with proteolytic enzymes and by physical separation, and

c₂) purifying the cells by physical separation and/or by immunoselection.

According to an advantageous embodiment of said methods, prior to step c₁ or c₂, they comprise an additional step of culturing the cells in a semi-solid medium containing suitable growth factors and/or cytokines.

A subject of the present invention is also a method for preparing isolated and purified cells able to differentiate into skeletal muscle cells, which method is characterized in that it comprises at least the following steps:

a₃) taking a sample of extramedullary adipose tissue,

b₃) isolating the cellular fraction of the vascular stroma, preferably by digestion of the extra-cellular matrix with proteolytic enzymes and by physical separation, and

c₃) culturing the cells in a semi-solid medium containing suitable growth factors and/or cytokines, and

d₃) purifying the cells by physical separation and/or by immunoselection.

According to an advantageous embodiment of said methods, they comprise an additional step d₁), d₂) or e₃) of expansion of the cells in vitro.

Advantageously, the physical separation is carried out by difference in adhesion onto a solid support or by difference in density, and the immunoselection is carried out using at least one antibody specific for a marker expressed by said cells (positive selection) and/or at least one antibody specific for a marker absent from said cells (negative selection) as defined above.

Advantageously, to implement the methods for obtaining the isolated and purified cells according to the invention:

the sample can be taken (steps a₁, a₂ or a₃) from a readily accessible adipose deposit, such as a subcutaneous adipose deposit,

the cellular fraction of the vascular stroma is isolated (step b₁, b₂ or b₃) by difference in density, in particular according to the protocol described by Bjorntorp et al. (mentioned above),

the culturing of the cells in a semi-solid medium containing suitable growth factors and/or cytokines (additional steps or step c₃) is carried out in a medium containing methylcellulose supplemented with fetal calf serum, bovine serum, insulin, transferrin, SCE, IL3 and IL6,

the purification of the cells (step c₁, c₂ or d₃) is carried out either by separation on any suitable support (difference in adhesion) or else by centrifugation in a suitable gradient or by elutriation (difference in density), or by immuno-selection, according to conventional immunocyto-chemistry techniques, in particular techniques for (positive or negative) sorting of immunolabeled cells by flow cytometry or using magnetic beads, as described, for example, in Current protocols in Immunology, (John E. Coligan, 2000, Wiley and son Inc, Library of Congress, USA); at least one antibody specific for a marker expressed by said cells (positive selection) and/or at least one antibody specific for a marker absent from said cells (negative selection) as defined above are used for the cell sorting;

the expansion of the cells in vitro (step d₁, d₂ or e₃) is carried out in a suitable culture medium, such as for example, but in a nonlimiting manner, a DMEM F12 medium comprising either fetal calf serum or a plant substitute serum.

Compared to existing means for regenerating hematopoietic lines or for regenerating cardiac and skeletal muscle tissues, the cellular fractions and the isolated cells, and also the methods for the preparation thereof, as defined above, have the following advantages:

technical advantages

ease of sampling,

very large amount of tissue and of cells with possible expansion of the cells sampled, favorable to homologous or heterologous transplantation,

possibility of maintaining and multiplying, or even of immortalizing, the cells in vitro in a defined medium, favorable to homologous or heterologous transplantation,

possibility of regenerating the blood population and/or the muscle tissue of several individuals from a single individual,

possibility of keeping the cells frozen,

transfectable cells,

cells with a high secretory capacity which may be used to release proteins of therapeutic interest,

cells suitable for the in vitro screening of a large amount of therapeutic molecules capable of modulating cardiac or skeletal muscle activity or hematopoietic activity.

economic advantages

reduced period of hospitalization (no conditioning or cytapheresis).

ethical advantages

relatively noninvasive sampling,

no use of embryonic tissues.

Besides the above arrangements, the invention also comprises other arrangements which will emerge from the following description, which refers to examples of implementation of the method which is the subject of the present invention and also to the attached drawings, in which:

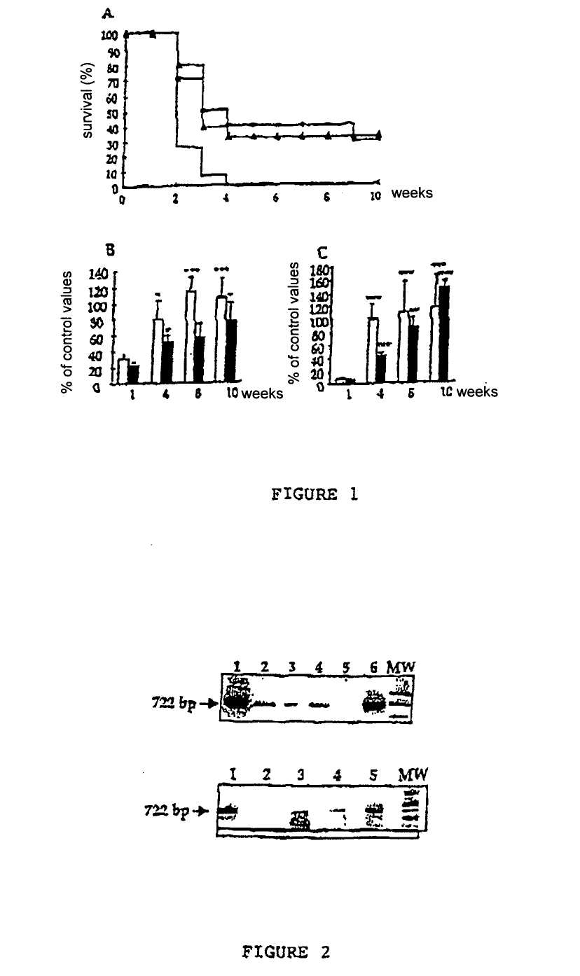

FIG. 1 illustrates the regeneration of hematopoietic lines, obtained by injection of cells of the vascular stroma of the extramedullary adipose tissue or of bone marrow cells (control).

FIG. 1A is a Kaplan-Meier graph representing the percentage survival of lethally irradiated mice (along the y-axis), over a period of 10 weeks following irradiation (along the x-axis). The non-regenerated irradiated mice are represented by circles, the mice given bone marrow cell transplants are represented by squares and the mice given vascular stroma cell transplants are represented by triangles. Each group comprises an initial number of 10 to 15 mice.

FIGS. 1B and 1C illustrate, respectively, the number of platelets and of leukocytes in irradiated mice regenerated with bone marrow cells (in black) or cells of the vascular stroma of adipose tissue (in white). The results are expressed as a percentage relative to the values for the nonirradiated controls, and the values indicated represent the mean±standard error, obtained on groups of 5 to 15 mice.

FIG. 2 illustrates the detection by PCR of the sry gene specific for chromosome Y in the spleen (upper panel) and the blood (lower panel) of regenerated female mice, performed 10 weeks after transplantation of bone marrow cells or of vascular stroma cells. Upper panel: the PCR is performed on 50 ng (lines 1, 5 and 6) or 150 ng (lines 2 to 4) of spleen DNA. A 722 bp product is detected in the mice regenerated with bone marrow cells (line 1) or vascular stroma cells (lines 2-4), derived from a male mouse. No signal is detected in the control female mice (line 5). A blood sample from a male mouse is used as a positive control (line 6). A molecular weight marker is indicated as a reference (MW). Lower panel: the PCR is carried out on 50 ng of blood DNA. A 722 bp product is detected in the animals regenerated with bone marrow cells (line 5) or vascular stroma cells (lines 3-4), derived from a male mouse. No signal is detected in the control female mice (line 2). A blood sample from a male mouse is used as a positive control (line 1). A molecular weight marker is indicated as a reference (MW).

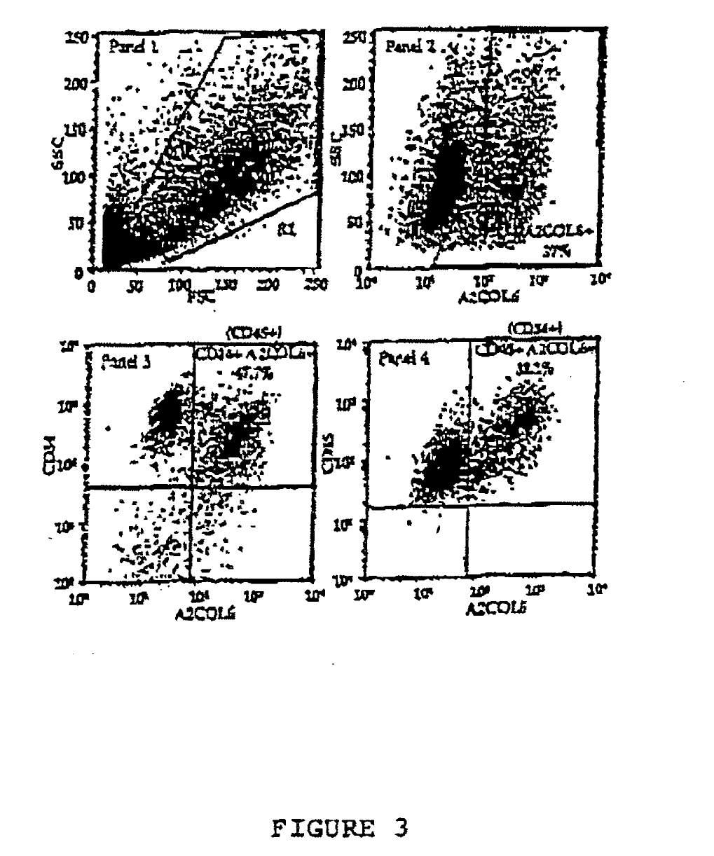

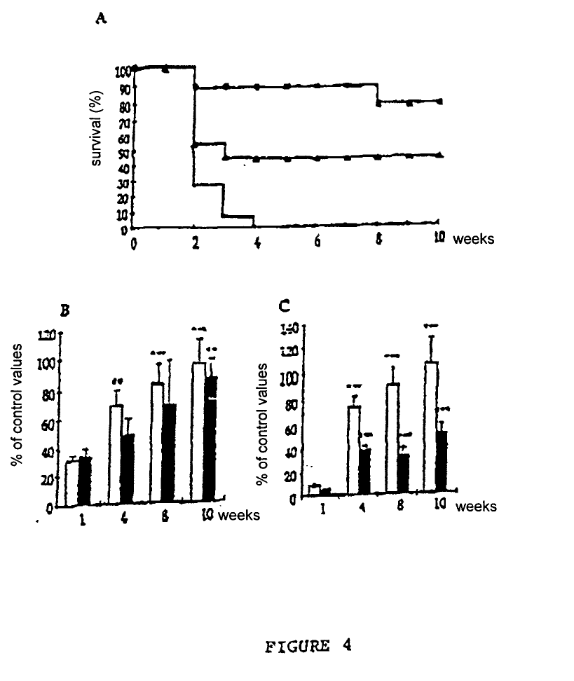

FIG. 3 illustrates the analysis by flow cytometry of the cells of the vascular stroma of male C57B1/6 mice. Panel 1: region R1 corresponds to the cell population selected for the analysis, as a function of the particle size parameters (FSC: forwards scatter), along the x-axis, and of the size parameters (SSC: side scatter), along the y-axis. Panel 2: distribution of the cells positive for the A2COL6 antigen specific for preadipocytes (along the x-axis) as a function of cell size (along the y-axis). Panels 3 and 4: representation of a triple labeling for the preadipocyte-specific antigen (A2COL6) and for two antigens specific for hematopoietic stem cells (CD45 and CD34). Panel 3 represents the distribution of the A2COL6⁺ (along the x-axis) and CD34⁺ (along the y-axis) cells in the CD45⁺ cell population. Panel 4 represents the distribution of the A2COL6⁺ (along the x-axis) and CD45⁺ (along the y-axis) cells in the CD34⁺ cell population.

FIG. 4 illustrates the regeneration of hematopoietic lines, obtained by injection of the preadipocyte line 3T3-L1 or of bone marrow cells (control). FIG. 1A is a Kaplan-Meier graph representing the percentage survival of the lethally irradiated mice (along the y-axis), over a period of 10 weeks following irradiation (along the x-axis). The non-regenerated irradiated mice are represented by circles, the mice given bone marrow cell transplants by squares and the mice given 3T3-L1 preadipocyte line transplants by triangles. Each group comprises an initial number of 10 to 15 mice.

FIGS. 4B and 4C represent, respectively, the number of platelets and of leukocytes in irradiated mice regenerated with bone marrow cells (in white) or with the 3T3-L1 preadipocyte line (in black). The results are expressed as a percentage relative to the values for the nonirradiated controls, and the values indicated represent the mean±standard error, obtained on groups of 5 to 15 mice.

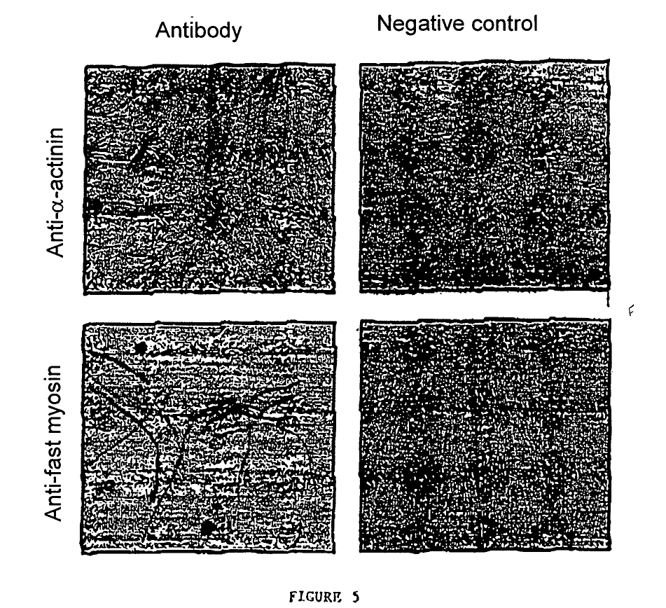

FIG. 5 illustrates the analysis by immunocyto-chemistry of the differentiation into cardiomyocytes and into skeletal muscle cells of the cells isolated from the vascular stroma of the extra-medullary adipose tissue, according to the methods of the invention. Upper panels: the presence of differentiated cardiomyocytes and differentiated skeletal muscle cells is detected specifically using an anti-α-actinin antibody (left panel); by comparison, in the negative control, no labeling is observed in the absence of anti-α-actinin antibody (right panel). Lower panels: the presence of differentiated skeletal muscle cells is detected specifically using an antibody against rapid isoforms of myosin (left panel); by comparison, in the negative control, no labeling is observed in the absence of antibody against rapid isoforms of α-myosin (right panel).

### EXAMPLE 1

### Materials and Methods

### 1) Isolation of Bone Marrow Cells

The bone marrow cells are isolated from femurs of 6-week-old male C57B1/6 mice; the red blood cells are removed by treatment with a solution of 9‰ ammonium chloride in water, and the cells are then centrifuged at 600 g for 10 minutes and resuspended in PBS, before being counted and injected.

### 2) Isolation of the Vascular Stroma Cells (Stroma-Vascular Fraction or SVF)

The cells are isolated according to the protocol described by Björntorp et al., mentioned above. More precisely, the inguinal adipose tissue is taken from 6-week-old male C57B1/6 mice and digested at 37° C. for 45 min, in a PBS buffer containing 0.2% of BSA and 2 mg/ml of collagenase. The digestion product is filtered successively through a 100 μm and 25 μm filter, and is then centrifuged at 800 g for 10 minutes; the stromal cells thus isolated are resuspended in PBS buffer and then counted and used in transplant experiments or for immunoanalyses.

### 3) Culturing of the Preadipocyte Line

The mouse preadipocyte line 3T3-L1 (ATCC reference CL-173) is cultured in DMEM medium containing 10% of heat-inactivated fetal calf serum and 2 mM of L-glutamine. The confluent 3T3-L1 cell cultures are harvested by trypsinization, counted and used for transplant experiments or immunoanalyses.

### 4) Transplantation of Cells (Bone Marrow, Vascular Stroma and 3T3-L1 Line)

On the day of transplantation, 8- to 10-week-old female C57B/6 mice are lethally irradiated at 10 Gy, in a single dose, and are then injected with 5×10⁶ to 10⁷ cells, in a volume of 400 μl, intravenously in the tail vein or intraperitoneally. The mice are fed with acidified water and autoclaved food. The animals are handled in accordance with the directives relating to animal experimentation.

### 5) Hematological Analysis

4, 8 or 10 weeks after transplantation, a 200 μl sample of peripheral blood is taken from the retro-orbital plexus of the mice given transplants, and immediately transferred into a tube containing heparin. Peripheral blood samples taken from nonirradiated mice are used as a positive control and peripheral blood samples taken from nonregenerated irradiated mice are used as a negative control. The counting of total blood cells and the proportion of the various types of nuclear cells is performed automatically with a hematological analysis device.

### 6) Analysis by Polymerized Chain Reaction (PCR)

10 weeks after transplantation, the total genomic DNA is extracted from the cells of hematopoietic tissues (bone marrow, spleen, thymus, liver) and of the blood, according to the conventional techniques described in Current Protocols in Molecular Biology, (1990-2000), John Wiley and Sons, Inc. New York. The DNA samples are amplified in a volume of 50 μl containing 20 pmol of each of the primers for the sry gene, specific for the Y chromosome, according to the protocol described in Pang et al., mentioned above.

From the DNA of the mice given transplants with bone marrow cells or vascular stroma cells, a 722 bp fragment corresponding to positions 256 to 978 of the sry gene is obtained.

From the DNA of the mice given transplants with the 3T3-L1 line, various fragments, which do not correspond to the sry gene, but the profile of which is specific for these cells, are obtained.

For each amplification series, samples originating from male and female mice are used as a positive and negative control, respectively.

### 7) Immunochemical Analysis

The cells in suspension, isolated as described in Example 1.2, are incubated with a first antibody, anti-CD34 coupled to biotin (Clinisciences) or anti-CD45 (Clinisciences), diluted in PBS buffer containing 0.1% of BSA. After washes and centrifugations, the cells are incubated respectively with a secondary antibody [anti-mouse immunoglobulins coupled to fluorescein isothiocyanate (A2COL6), anti-rat immunoglobulins coupled to Texas red (CD45)] and with streptavidin coupled to Cy-chrome, according to the conventional protocols described in Current Protocols in Molecular Biology, mentioned above. The cells are then fixed in PBS buffer containing 0.037% of para-formaldehyde and analyzed by flow cytometry, or else they are fixed on cover slips by centrifugation and observed by fluorescence microscopy.

### 8) Hematopoietic Differentiation

The short-term hematopoietic progenitors or precursors are analyzed using the hematopoietic tissues of female C57 B1/6 mice which have been lethally irradiated and then given transplants, according to the protocol described in Example 1.4.

The long-term hematopoietic progenitors or precursors are analyzed using the hematopoietic tissues of SCID mice given a nonlethal irradiation of 4 Gy and then given transplants, according to the protocol described in Example 1.4.

### a) Lymphoid Lines

Thymocytes (lymphocyte precursors) are purified from the thymus of the female mice given transplants, according to conventional techniques as described in Current Protocols in Immunology (John E. Coligan, 2000, Wiley and Son Inc, Library of Congress, USA). The total genomic DNA is then extracted from the thymocytes and amplified as described in Example 1.6.

### b) Myeloid Lines

Extracts of bone marrow and spleen cells from the mice given transplants are prepared according to conventional techniques as described in Current Protocols in Immunology (John E. Coligan, 2000, Wiley and Son Inc, Library of Congress, USA), and the cells are then seeded in a medium containing 1% methyl-cellulose, 15% fetal calf serum, 1% of bovine serum, 10 μg/ml of human insulin, 200 μg/ml transferrin, 10⁻⁴ M mercaptoethanol, 2 mM L-glutamine, 50 ng/ml recombinant murine SCF, 10 ng/ml recombinant murine IL3 and 10 ng/ml recombinant human IL6 (medium METHOCUL™ GF M3534, STEM CELL TECHNOLOGIES INC), according to the manufacturer's instructions.

### 9) Muscle Differentiation

Cells of the vascular stroma, isolated as described in Example 1.2, are cultured in the methylcellulose-based semi-solid medium as defined above (Example 1.8).

The cardiomyocytes and the skeletal muscle cells are detected by the expression of α-actinin, which is revealed using specific antibodies (clone EA-53, SIGMA), according to the manufacturer's instructions.

The skeletal muscle cells are detected specifically by the expression of the heavy chain of the myosin isoform (rapid isoforms), which is revealed using specific antibodies (clone MY-32, SIGMA), according to the manufacturer's instructions.

The cardiomyocytes are also detected by their spontaneous contractile activity in the presence or absence of agonists or antagonists of muscarinic acetylcholine receptors (carbamylcholine and atropine, respectively) or β-adrenergic receptors (isoproterenol and propranolol, respectively).

More precisely, 1 ml of DMEM medium containing carbamylcholine (2 μM), atropine (10 μM), isoproterenol (10 μM) or propranolol (40 μM) is added to the methyl-cellulose-based medium. After incubation for 5 min, necessary for diffusion of the molecules, the excess buffer is removed and the cell contractions are counted under the microscope for one minute.

### EXAMPLE 2

### Regeneration of Hematopoietic Lines from Bone Marrow Cells (Control) or from Cells of the Vascular Stroma, in Lethally Irradiated Female Mice

The recipient mice are irradiated and then given transplants, intraperitoneally, with cells of the vascular stroma or else with bone marrow cells (control), according to the protocols described in Example 1.

### 1) Survival of the Irradiated Animals

FIG. 1A illustrates the survival of the animals analyzed 10 weeks after irradiation, so as to evaluate the long-lasting regeneration of hematopoietic lines. The results observed show that the nonregenerated mice die within the 3 weeks following irradiation. On the other hand, a 40% survival is observed among the animals given transplants with cells of the vascular stroma or else with bone marrow cells. Given that the lethal irradiation eliminates most of the endogenous hematopoietic precursors, the results observed indicate that the survival of the regenerated animals is related to the transplanted cells.

### 2) Analysis of Hematopoietic Lines

FIGS. 1B and 1C illustrate the regeneration of the various hematopoietic lines, expressed as percentage relative to the values for the nonirradiated control.

In the nonregenerated mice, the number of platelets falls rapidly in one week, from an initial value of 551×10³ platelets/μl to a value of 145±6×10³ platelets/μl. On the other hand, in the mice given transplants with cells of the vascular stroma or bone marrow cells, the number of platelets gradually increases to reach significant values at 4 weeks which are virtually equal to those of the control at 10 weeks (FIG. 1C).

In the mice regenerated with the cells of the vascular stroma, the leukocytes are almost undetectable one week after irradiation, but 7 weeks later, they return to values identical to that of the control.

FIGS. 1B and 1C also show that the restoring of the number of platelets and leukocytes is more rapid in the mice regenerated with the bone marrow cells.

The analysis of the leukocyte population shows that, in the mice regenerated with bone marrow cells or cells of the vascular stroma, the proportions of lymphocytes, of monocytes and of granulocytes are equivalent to those of the nonirradiated control mice.

Consequently, these results demonstrate that intra-peritoneal injection of cells of the vascular stroma makes it possible to keep alive lethally irradiated mice and makes it possible to regenerate the myeloid and lymphoid lines with an efficiency comparable to that observed with an equivalent number of bone marrow cells, but with a delay of a few weeks.

### 3) Demonstration of the Transplanted Cells in the Recipient Mice

The presence of male cells derived from the injected cells in the hematopoietic tissues and the blood of the regenerated female mice was analyzed by PCR using primers for the sry gene, specific for the Y chromosome.

No male cell is detected in the group of nonregenerated mice or in the control female mice.

On the other hand, a 722 bp product specific for the sry gene is present in a very large amount in the hematopoietic tissues (bone marrow, thymus, spleen) and in the blood of the mice injected with bone marrow cells derived from male mice (FIG. 2). A product of identical size is also detected in the hematopoietic tissues (bone marrow, thymus, spleen) and in the blood of the female mice 10 weeks after transplantation of cells of the vascular stroma, derived from male mice (FIG. 2).

Consequently, the results given in FIG. 2 show that some cells of the vascular stroma have the ability to migrate from the peritoneal cavity to the hematopoietic sites, to proliferate and to differentiate into circulating blood cells, thus allowing regeneration of functional hematopoiesis in the lethally irradiated mice.

### 4) Analysis of the Hematopoietic Differentiation

The sry gene is detected in purified thymocytes (lymphocyte precursors) derived from the female mice given transplants with cells of the vascular stroma of adipose tissue from a male mouse, indicating that these cells have a potential for differentiation into lymphoid lines.

Clones of myeloid cells containing the sry gene are obtained from the cells of the bone marrow and of the spleen of the mice (C57 B1/6 and SCID) given transplants with cells of the vascular stroma of adipose tissue; in the nonlethally irradiated SCID mice, the number of hematopoietic clones containing the sry gene is significantly higher. These results indicate that the cells of the vascular stroma of adipose tissue contain hematopoietic progenitors capable of differentiating into myeloid lines.

This set of results indicates that the vascular stroma of adipose tissue contains short-term and long-term hematopoietic progenitors capable of differentiating into lymphoid and myeloid hematopoietic lines able to regenerate functional hematopoiesis in the irradiated mice.

### EXAMPLE 3

### Phenotypic Analysis of the Cells of the Vascular Stroma

As far as the vascular stroma consists of a heterogeneous cell population, flow cytometry immunolabeling experiments were carried out in order to identify the cells of the vascular stroma which have hematopoietic activity.

FIG. 3 shows that 35.3±3.6% of the population of cells of the vascular stroma express the A2COL6 antigen which is a marker specific for preadipocytes (panel 2). Using 2 antigens specific for hematopoietic stem cells, immunolabeling experiments indicate that 35.8±6% and 30.6%±3.1%, respectively, of the cells are positive for CD34 and CD45, which demonstrates that the cells of the vascular stroma, isolated from adipose tissues, are an unexpected source of hematopoietic stem cells able to differentiate into cells of the various hematopoietic lines (myeloid and lymphoid lines).

Complementary triple labeling experiments reveal that most of the A2COL6-positive cells also express the CD34 and CD45 antigens (FIG. 3: panels 3 and 4), which demonstrates that the preadipocytes can be considered to be hematopoietic precursors. This triple labeling was also obtained with the 3T3-L1 preadipocyte line.

### EXAMPLE 4

### Regeneration of Hematopoietic Lines from Cells of the 3T3-L1 Preadipocyte Line, in Lethally Irradiated Female Mice

The recipient mice are irradiated and then given transplants, intravenously or intraperitoneally, with cells of the 3T3-L1 preadipocyte line, according to the protocols described in Example 1.

Preliminary experiments show that the transplants are less efficient when the cells are injected intraperitoneally, probably due to the slowness of migration of the cells from the peritoneal cavity to the hematopoietic centers.

Consequently, the results of the intravenous injections are given in FIG. 4.

FIG. 4A shows that, 10 weeks after lethal irradiation, 80% of the mice given transplants with bone marrow cells are still alive, while only 50% of the mice given transplants with 3T3-L1 cells have survived the irradiation.

Blood cell counts for the two groups of mice given transplants show partial restoration of the number of platelets and leukocytes within the four weeks following irradiation (FIGS. 4B and 4C). At 10 weeks, the number of platelets again reaches values equivalent to those of the nonirradiated controls (FIG. 4B) for the two groups of mice given transplants. On the other hand, at 10 weeks, the mice regenerated with bone marrow cells again reach values equivalent to those of the nonirradiated controls, whereas the number of leukocytes does not exceed 50% of the values for the controls in the mice regenerated with 3T3-L1 cells.

Analysis of the leukocyte population shows that, in the mice regenerated with 3T3-L1 cells, the proportions of lymphocytes, of monocytes and of granulocytes are equivalent to that of the nonirradiated control mice.

Consequently, these results demonstrate that, compared to the bone marrow cells, which are more efficient and allow regeneration of the myeloid and lymphoid lines with values comparable to those of the nonirradiated controls, from 8 weeks after irradiation, the 3T3-L1 line makes it possible, however, to partially regenerate the hematopoietic lines.

### EXAMPLE 5

### In Vitro Differentiation of Cells of the Vascular Stroma of Adipose Tissue into Cardiac and Skeletal Muscle Cells

Cells of the vascular stroma, isolated as described in Example 1.2, are cultured and then analyzed under the conditions described in Example 1.9.

Under these conditions, multiplication of the cells is observed, followed by differentiation of the cells into cardiac and skeletal contractile cells.

FIG. 5 shows the presence of cardiomyocytes and of skeletal muscle cells characterized by the expression of α-actinin. It also shows specific detection of the skeletal muscle cells by expression of the heavy chain of the myosin isoform.

Table I below shows the spontaneous contractile activity of the cells which is specific for cardiomyocytes, and also the inhibition of the contractions with carbamylcholine (agonist of muscarinic acetylcholine receptors) and the reversal of its effect by adding atropine (antagonists of the same receptors). The values correspond to the mean of 3 independent measurements.

TABLE I

| Carbamylcholine | − | + | + |
|---|---|---|---|
| Atropine | − | − | + |
| Contractions | 100% | 53% | 106% |

Table II below shows the stimulation of the contraction frequency with isoproterenol (agonist of β-adrenergic receptors) and the reversal of its effect by adding propranolol (antagonist of the same receptors). The values correspond to the mean of 3 independent measurements.

TABLE II

| Isoproterenol | − | + | + |
|---|---|---|---|
| Propranolol | − | − | + |
| Contractions | 100% | 160% | 100% |

As emerges from the above, the invention is in no way limited to its methods of implementation, preparation and application which have just been described explicitly; on the contrary, it encompasses all the variants thereof which may occur to a person skilled in the art, without departing from the context or scope of the present invention.

1. A cellular fraction of the vascular stroma of extramedullary adipose tissue, as a medicinal product.

2. The use of the cellular fraction of the vascular stroma of extramedullary adipose tissue, for preparing a medicinal product intended for the treatment of diseases in which medullary depletion is observed.

3. The use of the cellular fraction of the vascular stroma of extramedullary adipose tissue, for preparing a medicinal product intended for the treatment of myopathies, of cardiomyopathies and of diseases in which muscle degeneration is observed.

4. An isolated and purified cell able to regenerate hematopoietic lines, characterized in that it is isolated from the cellular fraction of the vascular stroma of extramedullary adipose tissue.

5. An isolated and purified cell able to differentiate into a cardiomyocyte, characterized in that it is isolated from the cellular fraction of the vascular stroma of extramedullary adipose tissue.

6. The cells as claimed in claim 4 or claim 5, characterized in that they can be obtained by the following successive steps:
taking a sample of extramedullary adipose tissue,
isolating the cellular fraction of the vascular stroma, preferably by digestion of the extracellular matrix with proteolytic enzymes and by physical separation, and
purifying the cells by physical separation and/or by immunoselection.

7. The cells as claimed in claim 6, characterized in that the purification step is preceded by an additional step of culturing the cells in a semi-solid medium containing suitable growth factors and/or cytokines.

8. The cells as claimed in any one of claims 4, 6 or 7, characterized in that they express at least one marker for adipocyte stem cells or precursors and/or at least one marker for hematopoietic stem cells or precursors.

9. The cells as claimed in any one of claims 5, 6 or 7, characterized in that they express at least one marker for adipocyte stem cells or precursors and/or at least one marker for cardiomyocyte stem cells or precursors.

10. The cells as claimed in claim 8 or claim 9, characterized in that said marker for adipocyte precursors is selected from the group consisting of A2COL6/pOb24, LPL and Pref-1.

11. The cells as claimed in claim 8, characterized in that said marker for hematopoietic stem cells or precursors is selected from the group consisting of: CD34, CD45, Thy-1, Sca-1, CD117 and CD38.

12. The cells as claimed in claim 9, characterized in that said marker for cardiomyocyte stem cells or precursors is selected from the group consisting of α-actinin and the GATA-4 factor.

13. The cells as claimed in claim 10 or claim 11, characterized in that they express at least A2COL6/pOb24, CD34 and CD45.

14. The cells as claimed in any one of claims 4 to 13, characterized in that they are of human origin.

15. A modified cell, characterized in that it consists of a cell as claimed in any one of claims 4 to. 13, which has been genetically modified.

16. The modified cell as claimed in claim 15, characterized in that it comprises at least one mutation of an autologous gene.

17. The modified cell as claimed in claim 15, characterized in that it contains at least one copy of a heterologous gene.

18. The cells as claimed in any one of claims 15 to 17, characterized in that they are of human origin.

19. An immortalized cell line derived from the human cells as claimed in claim 14 or claim 18.

20. A medicinal product intended to regenerate hematopoietic lines, characterized in that it comprises cells as claimed in claim 4 or else modified cells or lines derived from these cells as claimed in any one of claims 15 to 19, and at least one pharmaceutically acceptable vehicle.

21. A medicinal product intended to regenerate the myocardium, characterized in that it comprises cells as claimed in claim 5 or else modified cells or lines derived from these cells as claimed in any one of claims 15 to 19, and at least one pharmaceutically acceptable vehicle.

22. The use of the cells as claimed in claim 4 or else of the modified cells or of the lines derived from these cells as claimed in any one of claims 15 to 19, for preparing a medicinal product intended for the treatment of diseases in which induced or constitutive medullary depletion is observed.

23. The use of the cells as claimed in claim 5 or else of the modified cells or of the lines derived from these cells as claimed in any one of claims 15 to 19, for preparing a medicinal product intended for the treatment of cardiomyopathies and of diseases associated with cardiac muscle degeneration.

24. A method for preparing the isolated and purified cells able to regenerate hematopoietic lines as defined in claim 4, which method is characterized in that it comprises at least the following steps:
a₁) taking a sample of extramedullary adipose tissue,
b₁) isolating the cellular fraction of the vascular stroma, preferably by digestion of the extra-cellular matrix with proteolytic enzymes and by physical separation, and
c₁)purifying the cells by physical separation and/or by immunoselection.

25. A method for preparing the isolated and purified cells able to differentiate into cardiomyocytes as defined in claim 5, which method is characterized in that it comprises at least the following steps:
a₂) taking a sample of extramedullary adipose tissue,
b₂)isolating the cellular fraction of the vascular stroma, preferably by digestion of the extra-cellular matrix with proteolytic enzymes and by physical separation, and
c₂)purifying the cells by physical separation and/or by immunoselection.

26. The method as claimed in claim 24 or claim 25, characterized in that, prior to step c₁ or c₂, it comprises an additional step of culturing the cells in a semi-solid medium containing suitable growth factors and/or cytokines.

27. A method for preparing isolated and purified cells able to differentiate into skeletal muscle cells, which method is characterized in that it comprises at least the following steps:
a₃) taking a sample of extramedullary adipose tissue,
b₃) isolating the cellular fraction of the vascular stroma, preferably by digestion of the extra-cellular matrix with proteolytic enzymes and by physical separation, and
c₃) culturing the cells in a semi-solid medium containing suitable growth factors and/or cytokines, and
d₃) purifying the cells by physical separation and/or by immunoselection.

28. The method as claimed in any one of claims 24 to 26, characterized in that it comprises an additional step d₁), d₂) or d₃) of expansion of the cells in vitro.

29. The use of the cells as claimed in claim 4 or else of the modified cells or of the lines derived from these cells as claimed in any one of claims 15 to 19, for screening molecules capable of modulating hematopoietic activity.

30. The use of the cells as claimed in claim 5 or else of the modified cells or of the lines derived from these cells as claimed in any one of claims 15 to 19, for screening molecules capable of modulating cardiac muscle activity.
